(19) United States
(12) Patent Application Publication
(10) Pub. No.: US 2024/0060032 A1
(43) Pub. Date: Feb. 22, 2024

(54) BIOSAMPLE PROCESSING APPARATUS AND BIOSAMPLE PROCESSING METHOD

(71) Applicants: CANON MEDICAL SYSTEMS CORPORATION, Otawara-shi (JP); Canon Kabushiki Kaisha, Tokyo (JP)

(72) Inventor: Katsuaki DEGUCHI, Otawara (JP)

(73) Assignees: CANON MEDICAL SYSTEMS CORPORATION, Otawara-shi (JP); Canon Kabushiki Kaisha, Tokyo (JP)

(21) Appl. No.: 18/450,845

(22) Filed: Aug. 16, 2023

(30) Foreign Application Priority Data
Aug. 18, 2022 (JP) 2022-130597

(51) Int. Cl. C12M 1/00

(52) U.S. Cl. CPC C12M 47/02

(57) ABSTRACT

A biosample processing apparatus of an embodiment includes a sample supply part, a first collection part, a separation part, and a second collection part. The sample supply part is connected to one end of a tubular flow path and supplies a first sample containing components extracted from a subject to the flow path. The first collection part is provided on the flow path and collects target cells to be cultured from the first sample supplied to the flow path. The separation part is provided downstream of the first collection part on the flow path and separates a blood plasma component or a blood serum component as a second sample from the first sample from which the target cells have been collected by the first collection part. The second collection part is connected to another end of the flow path and collects the second sample separated by the separation part.

### CROSS-REFERENCE TO RELATED APPLICATIONS

This application is based upon and claims the benefit of priority from Japanese Patent Application No. 2022-130597, filed on Aug. 18, 2022, the entire contents of which are incorporated herein by reference.

### FIELD

Embodiments described herein relate generally to a biosample processing apparatus and a biosample processing method.

### BACKGROUND

Stem cells such as induced pluripotent stem cells (iPS cells) and embryonic stem cells (ES cells) have the ability to differentiate into cells with various functions and are expected to be applied in the field of regenerative medicine, the field of drug discovery, and the like. For this reason, attention has been focused on cell culture technology for culturing various types of cells.

Conventionally, in general cell culture, substances derived from other animal species are added to culture media or the like in order to provide the cells to be cultured with essential components for cell culture.

### BRIEF DESCRIPTION OF THE DRAWINGS

FIG. 1 is a schematic diagram of an example of the configuration of a biosample processing apparatus according to an embodiment;

FIG. 2 is a flowchart of an example of processing by the biosample processing apparatus according to the embodiment;

FIG. 3 is a diagram illustrating an example of operations of the biosample processing apparatus according to the embodiment;

FIG. 4 is a diagram illustrating an example of the operations of the biosample processing apparatus according to the embodiment;

FIG. 5 is a diagram illustrating an example of the operations of the biosample processing apparatus according to the embodiment;

FIG. 6 is a diagram illustrating an example of the operations of the biosample processing apparatus according to the embodiment;

FIG. 7 is a diagram illustrating an example of the operations of the biosample processing apparatus according to the embodiment;

FIG. 8 is a diagram illustrating an example of the operations of the biosample processing apparatus according to the embodiment;

FIG. 9 is a schematic diagram of an example of the configuration of the biosample processing apparatus according to a first modification; and

FIG. 10 is a schematic diagram of an example of the configuration of the biosample processing apparatus according to the second modification.

### DETAILED DESCRIPTION

A biosample processing apparatus of an embodiment includes a sample supply part, a first collection part, a separation part, and a second collection part. The sample supply part is connected to one end of a tubular flow path and supplies a first sample containing components extracted from a subject to the flow path. The first collection part is provided on the flow path and collects target cells to be cultured from the first sample supplied to the flow path. The separation part is provided downstream of the first collection part on the flow path and separates a blood plasma component or a blood serum component as a second sample from the first sample from which the target cells have been collected by the first collection part. The second collection part is connected to another end of the flow path and collects the second sample separated by the separation part.

The following describes embodiments of a biosample processing apparatus and a biosample processing method in detail with reference to the accompanying drawings. The cell culture apparatus and the cell culture method according to the present application are not limited by the embodiments shown below. The embodiments can be combined with other embodiments and conventional technologies to the extent that there is no contradiction in description. In the following description, similar components are denoted by common symbols, and duplicated descriptions are omitted.

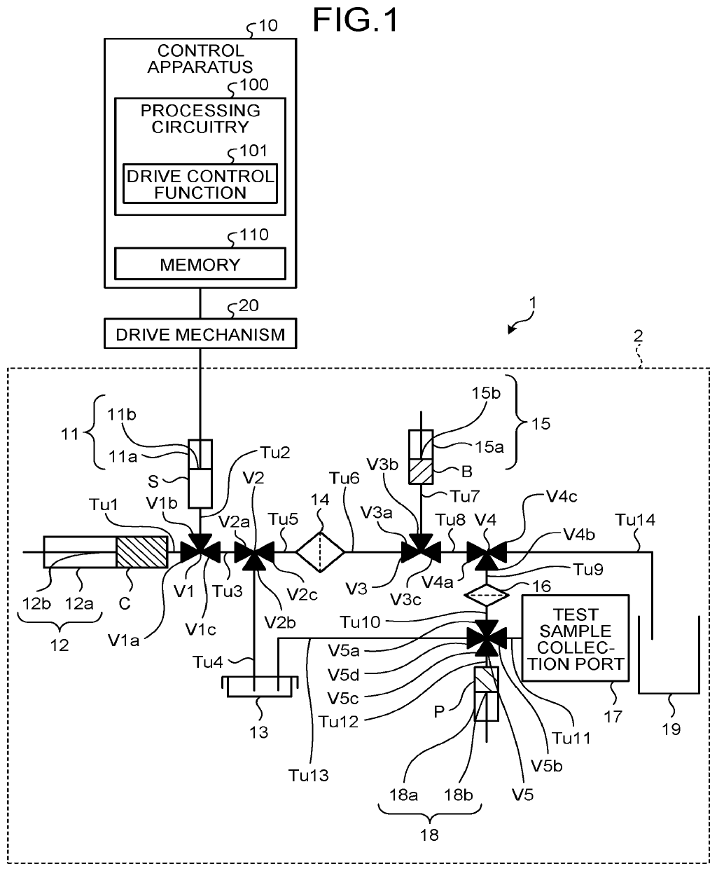

The following first describes the configuration of a biosample processing apparatus 1 according to the present embodiment. The biosample processing apparatus 1 is an apparatus automatically separating a target component to be cultured from a biosample while maintaining an aseptic condition inside. FIG. 1 is a schematic diagram of an example of the configuration of the biosample processing apparatus 1 according to the present embodiment.

The biosample processing apparatus 1 includes a control apparatus 10, a drive mechanism 20, and a biosample processing part 2. The biosample processing part 2 performs processing of collecting target cells from the biosample, removing unnecessary components, and the like.

The biosample processing part 2 includes a biosample supply part 11, a washing liquid supply part 12, a culture part 13, a cell capturing part 14, a reagent supply part 15, an unnecessary component removal part 16, a test sample collection port 17, a blood plasma component supply part 18, a waste liquid tank 19, branches V1 to V5, and tubes Tu1 to Tu9.

The biosample processing part 2 according to the present embodiment is maintained at a positive pressure condition. This condition makes it difficult for outside air to enter the biosample processing part 2. In addition, the biosample processing part 2 is a closed system being not directly exposed to the outside air, and thus the biosample processing part 2 can maintain its inside aseptic condition.

The control apparatus 10 is an information processing apparatus comprehensively controlling the entire biosample processing apparatus 1. The control apparatus 10 is connected to the drive mechanism 20. The configuration of the control apparatus 10 will be described below.

The biosample supply part 11 supplies a biosample S containing components extracted from a subject. The biosample supply part 11 is an example of the sample supply part. The biosample supply part 11 includes a syringe 11a and a piston 11b. The biosample supply part 11 is connected to the branch V1 by the tube Tu2. The biosample supply part 11 pushes out the biosample S in the syringe 11a by the piston 11b to supply the biosample S to the biosample processing part 2.

The syringe 11a and the piston lib may be replaced each time the biosample S is changed. In this case, the drive mechanism 20 includes a connection part capable of attaching and detaching the piston lib.

The washing liquid supply part 12 supplies a washing liquid C to the biosample processing part 2. The washing liquid supply part 12 includes a syringe 12a and a piston 12b. The washing liquid supply part 12 is connected to the branch V1 by the tube Tu1. The washing liquid supply part 12 pushes out the washing liquid C in the syringe 12a by the piston 12b to supply the washing liquid C to the biosample processing part 2.

The culture part 13 adjusts its internal environment to perform culture of target cells T separated from the biosample S. The culture part 13 is connected to the branch V2 by the tube Tu4. The culture part 13 is connected to the branch V5 by the tube Tu13.

The culture part 13 has a discharge mechanism (not illustrated) discharging an internal culture medium and the like to the waste liquid tank 19. The discharge mechanism can be implemented by, for example, a syringe and a piston or the like. When the culture medium and the like are discharged, the target cells T may move together with the culture medium and the like, but the target cells T can be captured by the cell capturing part 14 described below. Thus, the biosample processing apparatus 1 according to the present embodiment can easily perform an operation of discharging the culture medium performed in the case of culture medium replacement or the like.

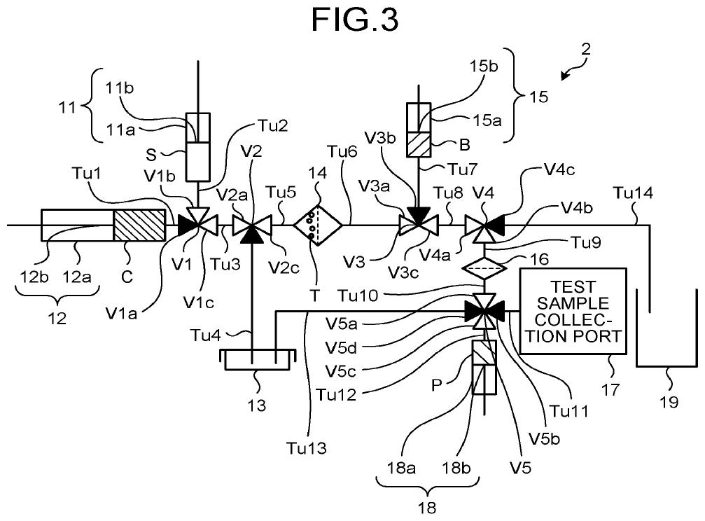

The cell capturing part 14 collects the target cells T (refer to FIG. 3 and the like) from the biosample S. The cell capturing part 14 is an example of the first collection part. For example, the cell capturing part 14 captures the target cells T and allows substances other than the target cells T to pass through. The cell capturing part 14 is implemented by a selective membrane, a filter, or the like. The cell capturing part 14 is connected to the branch V2 by the tube Tu5. The cell capturing part 14 is connected to the branch V3 by the tube Tu6.

The reagent supply part 15 supplies a reagent B to the culture part 13. The reagent supply part 15 includes a syringe 15a and a piston 15b. The reagent supply part 15 is connected to the branch V3 by the tube Tu7. The reagent supply part 15 pushes out the reagent B in the syringe 15a by the piston 15b to supply the reagent B to the culture part 13.

The unnecessary component removal part 16 separates a blood plasma component P from the biosample S from which the target cells T have been collected. For example, the unnecessary component removal part 16 removes unnecessary components such as a blood cell component from the biosample S from which the target cells T have been separated to separate the blood plasma component P. The unnecessary component removal part 16 is implemented by a filter or the like. The unnecessary component removal part 16 is connected to the branch V4 by the tube Tu9. The unnecessary component removal part 16 is connected to the branch V5 by the tube Tu10.

The test sample collection port 17 collects part of the separated blood plasma component P as a test sample. The test sample collection port 17 is connected to the branch V5 by the tube Tu11. For example, an empty sample tube or the like is connected to the test sample collection port 17. The test sample is collected to the sample tube or the like connected to the test sample collection port 17 by the blood plasma component supply part 18 described below.

For example, the test sample is analyzed by an automated biochemical analysis apparatus to measure various types of components such as albumin and electrolytes. In this case, the control apparatus 10 receives a measurement result from the automated biochemical analysis apparatus. The control apparatus 10 then performs control to supply the blood plasma component P in an amount corresponding to the measurement result to the culture part 13.

The blood plasma component supply part 18 supplies the blood plasma component P to the culture part 13. The blood plasma component supply part 18 includes a syringe 18a and a piston 18b. The blood plasma component supply part 18 is connected to the culture part 13 by the tube Tu13. The blood plasma component supply part 18 pushes out the blood plasma component P in the syringe 18a by the piston 18b to supply the blood plasma component P to the culture part 13.

The waste liquid tank 19 is a tank storing a waste liquid. The waste liquid tank 19 is connected to the branch V4 by the tube Tu14. For example, the waste liquid tank 19 stores the reagent B (for example, a used culture medium or the like), the washing liquid C, and the like.

The branch V1 selectively switches between a direction of flow from the biosample supply part 11 to the tube Tu3 and a direction of flow from the washing liquid supply part 12 to the tube Tu3.

The branch V1 includes valves V1a, V1b, and V1c. The valve V1a is connected to the washing liquid supply part 12 by the tube Tu1. The valve V1b is connected to the biosample supply part 11 by the tube Tu2. The valve V1c is connected to the branch V2 by the tube Tu3.

The branch V2 selectively switches between a direction of flow from the biosample supply part 11 to the cell capturing part 14 and a direction of flow from the cell capturing part 14 to the culture part 13.

The branch V2 includes valves V2a, V2b, and V2c. The valve V2a is connected to the valve V1c of the branch V1 by the tube Tu3. The valve V2b is connected to the culture part 13 by the tube Tu4. The valve V2c is connected to the cell capturing part 14 by the tube Tu5.

The branch V3 selectively switches between a direction of flow from the cell capturing part 14 to the unnecessary component removal part 16 and a direction of flow from the reagent supply part 15 to the cell capturing part 14.

The branch V3 includes valves V3a, V3b, and V3c. The valve V3a is connected to the cell capturing part 14 by the tube Tu6. The valve V3b is connected to the reagent supply part 15 by the tube Tu7. The valve V3c is connected to the branch V4 by the tube Tu8.

The branch V4 selectively switches between a direction of flow from the unnecessary component removal part 16 to the blood plasma component supply part 18 and a direction of flow from the unnecessary component removal part 16 to the waste liquid tank 19. The branch V4 is an example of a second switch part.

The branch V4 includes valves V4a, V4b, and V4c. The valve V4a is connected to the valve V3c by the tube Tu8. The valve V4b is connected to the unnecessary component removal part 16 by the tube Tu9. The valve V4c is connected to the branch V5 by the tube Tu10.

The branch V5 selectively switches among a direction of flow from the unnecessary component removal part 16 to the blood plasma component supply part 18, a direction of flow from the blood plasma component supply part 18 to the culture part 13, and a direction of flow from the blood plasma component supply part 18 to the test sample collection port 17. The branch V5 is an example of a first switch part.

The branch V5 includes valves V5a, V5b, V5c and V5d. The valve V5a is connected to the valve V4c by the tube Tu10. The valve V5b is connected to the test sample collection port 17 by the tube Tu11. The valve V5c is connected to the blood plasms component supply part 18 by the tube Tu12. The valve V5d is connected to the culture part 13 by the tube Tu13.

The drive mechanism 20 performs control to drive each part of the biosample processing apparatus 1. The drive mechanism 20 has a drive source such as a motor. The drive mechanism 20 is connected to a biosample syringe pump (not illustrated) driving the piston 11b of the biosample supply part 11. The drive mechanism 20 controls the biosample syringe pump to drive the piston 11b and to perform control to draw and supply the biosample S into the culture part 13.

The drive mechanism 20 is connected to a washing liquid syringe pump (not illustrated) driving the piston 12b of the washing liquid supply part 12. The drive mechanism 20 controls the washing liquid syringe pump to drive the piston 12b and to perform control to draw and supply the reagent B into the tube Tu1.

The drive mechanism 20 is connected to a syringe pump (not illustrated) driving the piston 15b of the reagent supply part 15. The drive mechanism 20 controls the syringe pump to drive the piston 15b and to perform control to draw and supply the reagent B into the culture part 13.

The drive mechanism 20 is connected to a blood plasma component syringe pump (not illustrated) driving the piston 18b of the blood plasma component supply part 18. The drive mechanism 20 controls the blood plasma component syringe pump to drive the piston 18b and to perform control to draw and supply the reagent B into the culture part 13.

For example, the drive mechanism 20 is connected to a valve drive apparatus (not illustrated) opening and closing the valves of the branches V1 to V5. The drive mechanism 20 controls the valve drive apparatus to perform the opening and closing control of the valve.

In the present embodiment, the drive mechanism 20 is configured to perform drive control of the various syringe pumps and the valve drive apparatus, but this is not limiting, and the control apparatus 10 may be configured to do so as well. In this case, the control apparatus 10 is connected to the various syringe pumps and the valve drive apparatus to control the drive of the piston and the opening and closing of the valve.

The following describes the configuration of the control apparatus 10. The control apparatus 10 has processing circuitry 100 and a memory 110. FIG. 1 illustrates only the processing circuitry 100 and the memory 110 in the control apparatus 10, but the control apparatus 10 may further include an input interface, a communication interface, a display (none of them is illustrated), and the like.

The processing circuitry 100 controls the operation of the biosample processing apparatus 1. For example, the processing circuitry 100 is implemented by a processor.

The processing circuitry 100 reads a computer program stored by the memory 110 and executes it to execute a drive control function 101 and a communication control function 102.

The drive control function 101 controls the drive mechanism 20 to operate each part of the biosample processing apparatus 1. For example, the drive control function 101 controls the drive mechanism 20 to operate the piston 11b of the biosample supply part 11 described below and to supply the biosample S such as blood to be processed to the biosample processing part 2.

In the present embodiment, it is assumed that the biosample S is blood with an anticoagulant and the biosample processing apparatus 1 separates the blood plasma component P, but blood without the anticoagulant may be used as the biosample S. In this case, the biosample processing apparatus 1 separates a blood serum component.

For example, the drive control function 101 controls the drive mechanism 20 to operate the piston 12b of the washing liquid supply part 12 described below and to supply the washing liquid C for washing the biosample processing part 2 to the biosample processing part 2. For example, a physiological saline solution or the like can be used as the washing liquid C.

For example, the drive control function 101 controls the drive mechanism 20 to operate the piston 15b of the reagent supply part 15 described below and to supply the reagent B such as the culture medium or a cell collection liquid to the culture part 13.

For example, the drive control function 101 controls the drive mechanism 20 to operate the piston 18b of the blood plasma component supply part 18 described below and to supply the blood plasma component P separated from the biosample S to the culture part 13. For example, the drive control function 101 controls the drive mechanism 20. The details of control processing of each part by the drive control function 101 will be described below.

The communication control function 102 controls communication between the biosample processing apparatus 1 and external apparatuses. For example, the communication control function 102 receives analysis data from an automated biochemical analysis apparatus (not illustrated) or the like via a communication interface.

The memory 110 stores therein various types of data. The memory 110 is connected to the processing circuitry 100. For example, the memory 110 stores therein various types of parameters such as the supply amount of the biosample S, the reagent B, and the like and the collection amount of the blood plasma component P. The memory 110 stores therein control information and the like of the drive mechanism 20 corresponding to the various types of parameters.

For example, the memory 110 stores therein various computer programs and the like being read and executed by the processing circuitry 100 to implement various types of functions. The memory 110 is implemented by a semiconductor memory element such as a random access memory (RAN) or a flash memory, a hard disk, an optical disk, or the like.

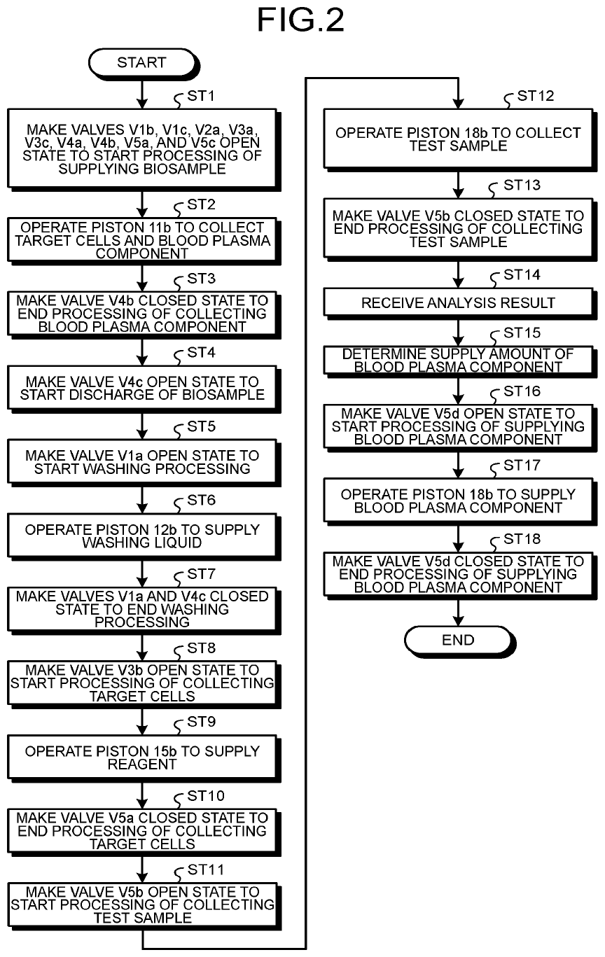

The following describes processing executed by the control apparatus 10 of the biosample processing apparatus 1 according to the present embodiment. FIG. 2 is a flowchart of an example of the processing executed by the control apparatus 10 of the biosample processing apparatus 1.

As a precondition, it is assumed that the syringe 11a of the biosample supply part 11 houses the biosample S, which is the blood of a donor with an anticoagulant. It is also assumed that the syringe 12a of the washing liquid supply part 12 houses the washing liquid C, which is a physiological saline solution. It is also assumed that the syringe 15a of the reagent supply part 15 houses the reagent B, which is a culture medium. It is also assumed that all the valves are in a closed state.

First, the drive control function 101 controls the drive of the drive mechanism 20 to make valves V1b, V1c, V2a, V3a, V3c, V4a, V4b, V5a, and V5c an open state to start processing of supplying the biosample S (Step ST1). Next, the drive control function 101 controls the drive of the drive mechanism 20 to operate the piston 11b of the biosample supply part 11 and to collect the target cells T and the blood plasma component P from the biosample S (Step ST2).

The following describes processing of collecting the target cells T and the blood plasma component P using FIG. 3. FIG. 3 is a diagram illustrating an example of operations of the biosample processing apparatus 1. The drive control function 101 operates the piston 11b to push out the biosample S to Tu2.

The pushed-out biosample S passes in order through the tubes Tu2, Tu3, and Tu5, the cell capturing part 14, the tubes Tu6, Tu8, and Tu9, the unnecessary component removal part 16, and the tubes Tu10 and Tu12. In this process, the target cells T are captured by the cell capturing part 14. The blood plasma component P is collected into the syringe 18a of the blood plasma component supply part 18.

The tubes Tu2, Tu3, Tu5, Tu6, Tu8, Tu9, Tu10, and Tu12 in this case are examples of the flow path. The syringe 18a collects the blood plasma component P and is thus an example of the second collection part.

Referring back to FIG. 2, the description continues. After a lapse of a certain period of time from the start of the processing of supplying the biosample S, the drive control function 101 makes the valve V4b a closed state to end the processing of collecting the blood plasma component P. (Step ST3). Next, the drive control function 101 makes the valve V4c an open state to start discharge of the biosample S from which the target cells T have been removed (Step ST4).

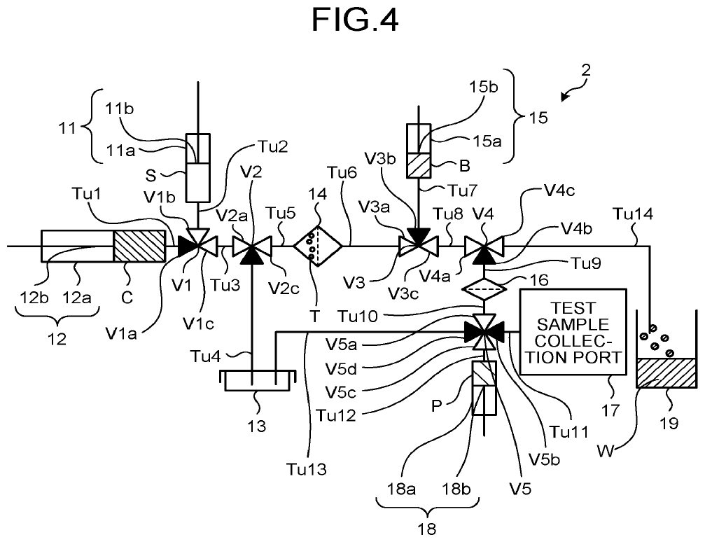

The following describes processing of discharging the unnecessary biosample S using FIG. 4. FIG. 4 is a diagram illustrating an example of the operations of the biosample processing apparatus 1. By making the valve V4c an open state, the unnecessary biosample S having passed through the cell capturing part 14 passes in order through the tubes Tu6, Tu8, and Tu14 to be discharged as a waste liquid W to the waste liquid tank 19.

Referring back to FIG. 2, the description continues. After the end of the processing of collecting the target cells T and the blood plasma component P, the drive control function 101 makes the valve V1a an open state to start washing processing (Step ST5). Next, the drive control function 101 operates the piston 12b of the washing liquid supply part 12 to supply the washing liquid (Step ST6).

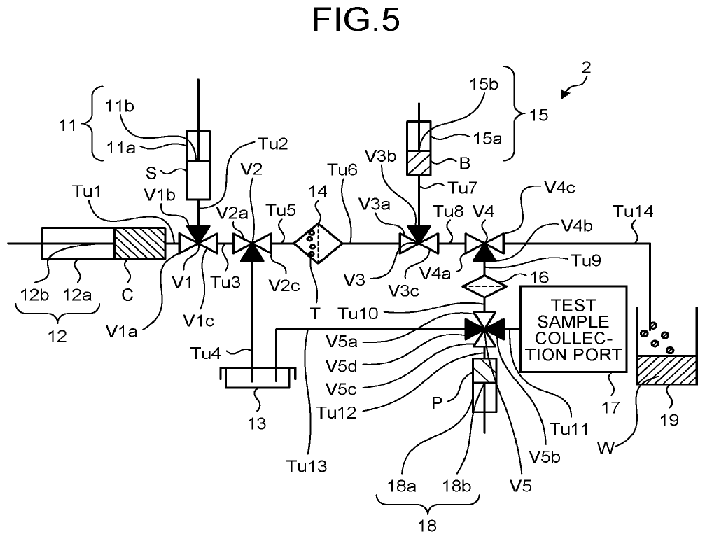

The following describes the washing processing of the biosample processing part 2. FIG. 5 is a diagram illustrating an example of the operations of the biosample processing apparatus 1. The drive control function 101 operates the piston 12b to push out the washing liquid C to Tu1. The pushed-out washing liquid C passes in order through the tubes Tu1, Tu3, and Tu5, the cell capturing part 14, and the tubes Tu6, Tu8, and Tu14 to be stored as the waste liquid W in the waste liquid tank 19.

By repeating the above operation a plurality of times, the biosample S inside the tubes Tu1, Tu3, Tu5, Tu6, Tu8, and Tu14 and the branches V1, V2, V3, and V4 of the biosample processing apparatus 1 is washed out. The target cells T are captured by the cell capturing part 14, and thus the target cells T do not flow to the waste liquid tank 19.

Referring back to FIG. 2, the description continues. After repeating the above washing operation, the drive control function 101 makes the valves V1a and V4c a closed state to end the washing processing (Step ST7). Next, the drive control function 101 makes the valve V3b an open state to start processing of collecting the target cells T (Step ST8). Next, the drive control function 101 operates the piston 15b of the reagent supply part 15 to start supply of the reagent B (Step ST9).

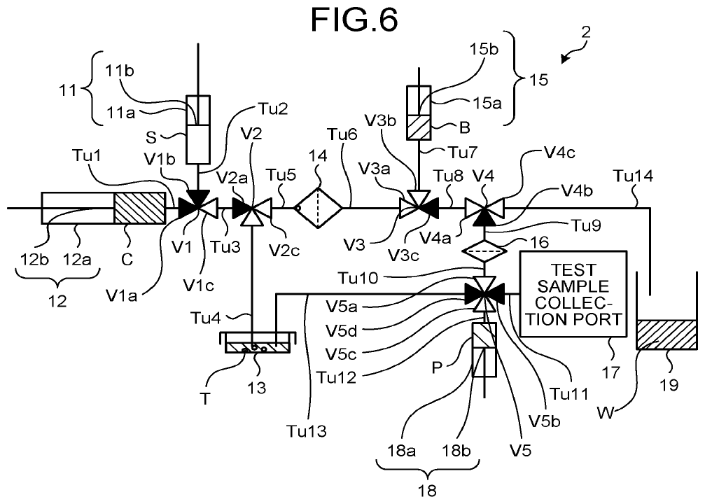

The following describes the processing of collecting the target cells T. FIG. 6 is a diagram illustrating an example of the operations of the biosample processing apparatus 1. The drive control function 101 operates the piston 15b to push out the reagent B to Tu7. The pushed-out reagent B passes in order through the tubes Tu7 and Tu6, the cell capturing part 14, and the tubes Tu5 and Tu4 to be supplied to the culture part 13. In this process, the target cells T captured by the cell capturing part 14 move together with the reagent B and are collected to the culture part 13.

Referring back to FIG. 2, the description continues. After collecting the target cells T, the drive control function 101 makes the valve V5a a closed state to end the processing of supplying the reagent B (Step ST10). Next, the drive control function 101 makes the valve V5b an open state to start processing of collecting the test sample (Step ST11) Next, the drive control function 101 operates the piston 18b of the blood plasma component supply part 18 to collect the test sample (Step ST12).

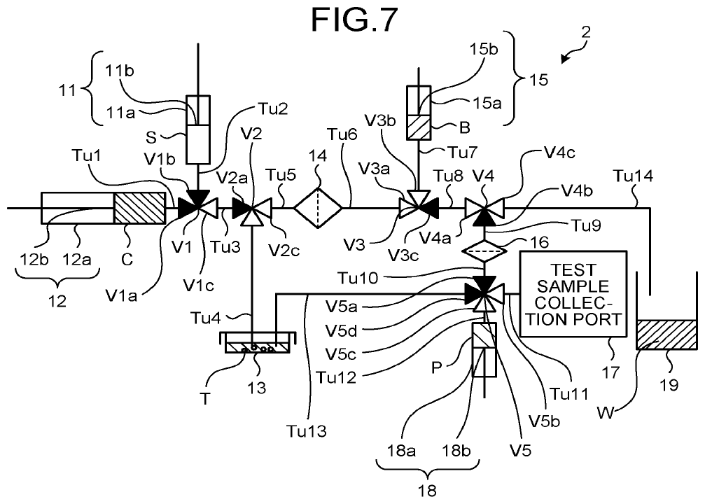

The following describes the processing of collecting the test sample. FIG. 7 is a diagram illustrating an example of the operations of the biosample processing apparatus 1. The drive control function 101 operates the piston 18b to push out the blood plasma component P to Tu2.

The pushed-out blood plasma component P passes in order through the tubes Tu11 and Tu12 and the test sample collection port 17 to be collected as the test sample into the empty sample tube connected to the test sample collection port 17. The test sample collection port 17 and the sample tube in this case collect the test sample and are thus examples of a third collection part.

In FIG. 7, described is a case in which the test sample collection processing is performed after the processing of collecting the target cells T, but the timing when the test sample collection processing is performed is not limited to this example. For example, it may be performed after the processing of collecting the blood plasma component P illustrated in FIG. 3, after the processing of discharging the unnecessary biosample S illustrated in FIG. 4, or the like.

Referring back to FIG. 2, the description continues. After collecting a certain amount of the blood plasma component P, the drive control function 101 makes the valve V5b a closed state to end the processing of collecting the test sample (Step ST13). Subsequently, the automated biochemical analysis apparatus performs analysis of the components of the collected test sample. Next, the communication control function 102 receives an analysis result from the automated biochemical analysis apparatus (Step ST14). The communication control function 102 in this case is an example of an acquisition part.

Next, the drive control function 101 determines a supply amount of the blood plasma component P based on the analysis result (Step ST15). In this case, the drive control function 101 determines the supply amount of the blood plasma component P in accordance with the analysis result. For example, the supply amount of the blood plasma component P is determined such that the concentration of a specific component such as albumin contained in the culture medium in the culture part 13 will be a certain concentration. The drive control function 101 in this case is an example of a determination part.

Referring back to FIG. 2, the description continues. After determining the supply amount of the blood plasma component P, the drive control function 101 makes the valve V5d an open state to start processing of supplying the blood plasma component P (Step ST16). Next, the drive control function 101 operates the piston 18b of the blood plasma component supply part 18 to supply the blood plasma component P (Step ST17).

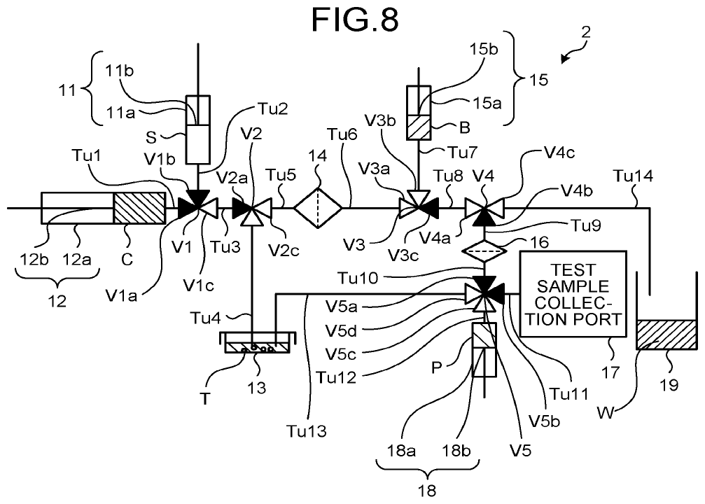

The following describes the processing of supplying the blood plasma component P. FIG. 8 is a diagram illustrating an example of the operations of the biosample processing apparatus 1. The drive control function 101 operates the piston 18b to push out the blood plasma component P to Tu2. The pushed-out blood plasma component P passes through the tubes Tu12 and Tu13 in this order to be supplied to the culture part 13.

Referring back to FIG. 2, the description continues. After supplying the determined amount of the blood plasma component P, the drive control function 101 makes the valve V5d a closed state, ends the processing of supplying the blood plasma component P, and ends this processing (Step ST18).

As described above, the biosample processing apparatus 1 according to the present embodiment collects the target cells T from the biosample S, removes unnecessary components such as a blood cell component from the biosample S from which the target cells T have been removed, and collects the blood plasma component P. Accordingly, both the target cells T and the blood plasma component P to be supplied to the culture part 13 can be obtained in single sample extraction. In other words, the biosample processing apparatus 1 according to the present embodiment can efficiently collect the target cells T and the component for use in the culture of the target cells T from the biosample S.

The biosample processing apparatus 1 according to the present embodiment includes the blood plasma component supply part 18 supplying the collected blood plasma component P to the culture part 13. Accordingly, the blood plasma component P essential for culture to the culture part 13 can be supplied. In addition, the target cells T and the blood plasma component P are derived from the same subject, and thus the possibility of rejection by irregular antibodies or the like occurring during culture can be reduced.

The biosample processing apparatus 1 according to the present embodiment includes the branch V4 switching between the collection of the biosample S from which the target cells T have been removed to the syringe 18a of the blood plasma component supply part 18 and the discharge thereof as the waste liquid W to the waste liquid tank 19. Accordingly, the biosample S from which the target cells T have been removed as the waste liquid W can be discharged without performing processing of removing unnecessary components after a required amount of the blood plasma component P is collected. In other words, the biosample processing apparatus 1 according to the present embodiment can efficiently process the biosample.

The biosample processing apparatus 1 according to the present embodiment includes the test sample collection port 17 for collecting part of the blood plasma component P collected to the syringe 18a as the test sample. Accordingly, analysis of a sample of the subject can be performed without separately extracting a sample from the subject.

The biosample processing apparatus 1 according to the present embodiment acquires an analysis result of the test sample and determines the supply amount of the blood plasma component P based on the analysis result. Accordingly, for example, the analysis result of the component essential for the culture of target cells T can be acquired, and thus the target cells T can be cultured after adjusting the concentration of the component to an appropriate concentration.

The embodiment described above can also be performed after being modified as appropriate by changing part of the configurations or functions of each apparatus. Thus, the following describes modifications according to the embodiment described above as other embodiments. The following mainly describes points different from the embodiment described above and omits detailed descriptions of points common to the details already described. The modifications described below may be implemented individually or implemented in combination as appropriate.

### FIRST MODIFICATION

In the embodiment described above, described is a mode in which the blood plasma component supply part 18 supplies the blood plasma component P to the culture part 13 under the control of the drive control function 101. However, the blood plasma component supply part 18 may supply the blood plasma component P directly to the culture medium.

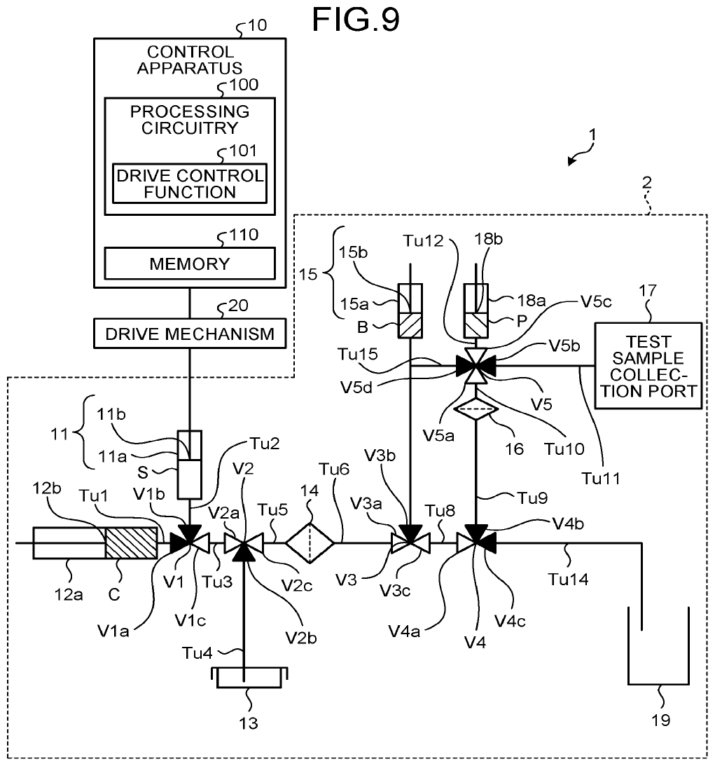

FIG. 9 is a schematic diagram of an example of the configuration of the biosample processing apparatus 1 according to a first modification. As illustrated in FIG. 9, in the biosample processing apparatus 1 according to the present modification, the valve V4a, the unnecessary component removal part 16, the test sample collection port 17, and the blood plasma component supply part 18 are provided, in the Y-axis direction of FIG. 9, not close to the culture part 13 or the waste liquid tank 19 but close to the biosample supply part 11 and the reagent supply part 15.

In addition, the tube Tu13 connecting the culture part 13 and the blood plasma component supply part 18 to each other is not provided, but a tube Tu15 connecting the tube Tu7 and the valve V4a to each other is provided. In the present modification, for example, when supplying the reagent B to the culture part 13, the drive control function 101 makes the valve V5d an open state, makes the valve V5a a closed state, and operates the piston 18b to push out the blood plasma component P in the syringe 18a.

The pushed-out blood plasma component P passes through the tube Tu12, is mixed with the reagent B supplied from the reagent supply part 15, and passes through the tubes Tu15, Tu7, and Tu6, the cell capturing part 14, and the tubes Tu5 and Tu4 to be supplied to the culture part 13.

Accordingly, the culture medium and the blood plasma component P can be mixed together before supplying them to the culture part 13, and thus the collection of the captured target cells T and the supply of the blood plasma component P can be performed simultaneously. In other words, the biosample processing apparatus 1 according to the present modification can reduce the risk of mixing of microorganisms and the like and the risk of rejection in cell culture more efficiently.

### SECOND MODIFICATION

In the embodiment described above, described is a mode in which the biosample processing apparatus 1 includes the test sample collection port 17. However, the biosample processing apparatus 1 does not necessarily include the test sample collection port 17.

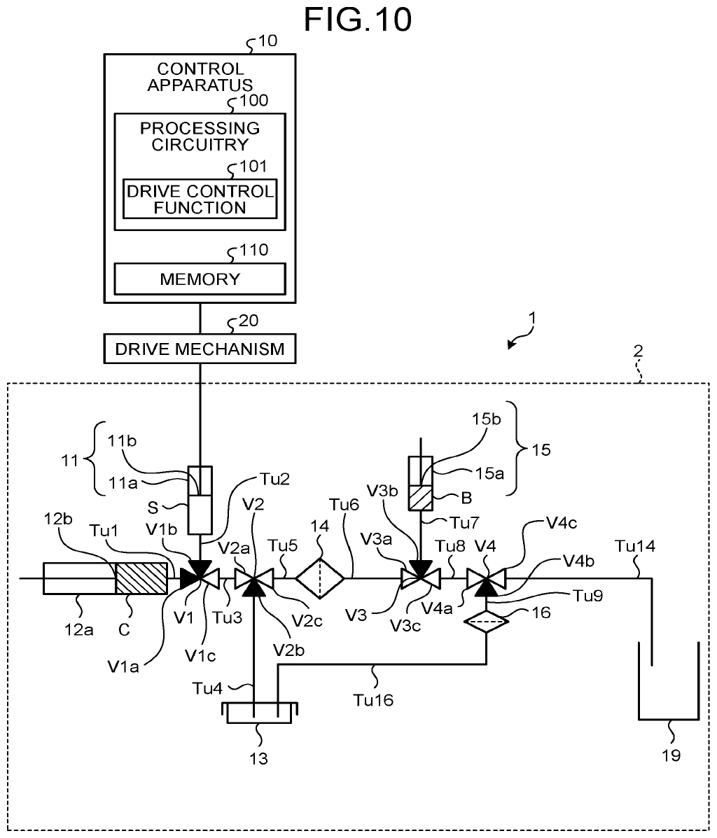

FIG. 10 is a schematic diagram of an example of the configuration of the biosample processing apparatus 1 according to a second modification. As illustrated in FIG. 10, in the biosample processing apparatus 1 according to the present modification, the unnecessary component removal part 16 and the culture part 13 are connected to each other directly by a tube Tu16. This configuration directly supplies the blood plasma component P from which unnecessary components have been removed to the culture part 13.

The biosample processing apparatus 1 according to the present modification can efficiently reduce the risk of mixing of microorganisms and the like and the risk of rejection in cell culture with a simple configuration.

### THIRD MODIFICATION

In the embodiment described above, described is a mode in which the drive control function 101 controls the drive mechanism 20 to control the operation of the pistons of the respective supply parts. However, the pistons of the respective supply parts may be operated by human hands. According to the biosample processing apparatus 1 according to the present modification, work related to biosample processing can be performed even in the event of a power failure or the like.

According to at least one embodiment described above, the target cells to be cultured and the component for use in the culture of the cells can be efficiently collected from the biosample.

The term “processor” used in the above description means, for example, a central processing unit (CPU), a graphics processing unit (GPU), or a circuit such as an application specific integrated circuit (ASIC) or a programmable logic device (for example, a simple programmable logic device (SPLD), a complex programmable logic device (CPLD), or a field programmable gate array (FPGA)).

Instead of storing the computer program in the memory, the computer program may be incorporated directly into a circuit of the processor. In this case, the processor reads the computer program incorporated into the circuit and executes it to implement functions. Each processor of the present embodiment is not limited to a case in which each processor is configured as a single circuit, but a plurality of independent circuits may be combined with each other to be configured as one processor to implement its functions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A biosample processing apparatus comprising:
a sample supply part connected to one end of a tubular flow path and configured to supply a first sample containing components extracted from a subject to the flow path;
a first collection part provided on the flow path and configured to collect target cells to be cultured from the first sample supplied to the flow path;
a separation part provided downstream of the first collection part on the flow path and configured to separate a blood plasma component or a blood serum component as a second sample from the first sample from which the target cells have been collected by the first collection part; and
a second collection part connected to another end of the flow path and configured to collect the second sample separated by the separation part.

2. The biosample processing apparatus according to claim 1, further comprising:
a culture part connected to a location between the sample supply part and the first collection part and connected to a location between the separation part and the second collection part; and
a first switch part provided at a connection part of the culture part between the separation part and the second collection part and configured to selectively switch between a direction of flow from the separation part to the second collection part and a direction of flow from the second collection part to the culture part.

3. The biosample processing apparatus according to claim 1, further comprising:
a tubular discharge path connected to the other end of the flow path and configured to discharge the first sample after the second sample is separated by the separation part; and
a second switch part provided at a connection part of the separation part between the flow path and the discharge path and configured to selectively switch between a direction of flow from the separation part to the second collection part and a direction of flow from the separation part to the discharge path.

4. The biosample processing apparatus according to claim 2, further comprising a third collection part connected to the first switch part and configured to collect part of the second sample as a test sample, wherein
the first switch part selectively switches among a direction of flow from the separation part to the second collection part, a direction of flow from the second collection part to the culture part, and a direction of flow from the separation part to the third collection part.

5. The biosample processing apparatus according to claim 4, further comprising:
an acquisition part configured to acquire an analysis result of components of the test sample; and
a determination part configured to determine a supply amount of the second sample to the culture part based on the analysis result.

6. The biosample processing apparatus according to claim 1, further comprising processing circuitry configured to
supply the first sample containing the components extracted from the subject from the sample supply part to the flow path,
collect the target cells to be cultured from the first sample supplied to the flow path to the first collection part,
separate the blood plasma component or the blood serum component as the second sample by the separation part from the first sample from which the target cells have been collected by the first collection part, and
collect the separated second sample to the second collection part.

7. The biosample processing apparatus according to claim 6, further comprising:
a culture part connected to a location between the sample supply part and the first collection part and connected to a location between the separation part and the second collection part; and
a first switch part provided at a connection part of the culture part between the separation part and the second collection part and configured to switch a direction of the flow path, wherein
the processing circuitry selectively switches the first switch part to a direction of flow from the separation part to the second collection part or a direction of flow from the second collection part to the culture part.

8. The biosample processing apparatus according to claim 6, the biosample processing apparatus further comprising:
a tubular discharge path connected to the other end of the flow path; and
a second switch part provided at a connection part of the separation part between the flow path and the discharge path, wherein
the processing circuitry
selectively switches the second switch part to a direction of flow from the separation part to the second collection part or a direction of flow from the separation part to the discharge path and
discharges the first sample after the second sample is separated from the discharge path when the second switch part is switched to the direction of flow from the separation part to the discharge path.

9. The biosample processing apparatus according to claim 7, further comprising a third collection part connected to the first switch part, wherein the processing circuitry
selectively switches the first switch part to a direction of flow from the second collection part to the culture part or a direction of flow from the separation part to the third collection part and
collects part of the second sample as a test sample to the third collection part when the first switch part is switched to the direction of flow from the separation part to the third collection part.

10. The biosample processing apparatus according to claim 9, wherein the processing circuitry
acquires an analysis result of components of the test sample and
determines a supply amount of the second sample to the culture part based on the analysis result.

11. A biosample processing method by a biosample processing apparatus including: a sample supply part connected to one end of a tubular flow path; a first collection part provided on the flow path; a separation part provided downstream of the first collection part on the flow path; and a second collection part connected to another end of the flow path, the biosample processing method comprising:
supplying a first sample containing components extracted from a subject from the sample supply part to the flow path;
collecting target cells to be cultured from the first sample supplied to the flow path to the first collection part;
separating a blood plasma component or a blood serum component as a second sample by the separation part from the first sample from which the target cells have been collected by the first collection part; and
collecting the second sample separated at the separating to the second collection part.

12. The biosample processing method according to claim 11, wherein the biosample processing apparatus further includes: a culture part connected to a location between the sample supply part and the first collection part and connected to a location between the separation part and the second collection part; and a first switch part provided at a connection part of the culture part between the separation part and the second collection part and configured to switch a direction of the flow path, and
the biosample processing method further comprises selectively switching the first switch part to a direction of flow from the separation part to the second collection part or a direction of flow from the second collection part to the culture part.

13. The biosample processing method according to claim 11, wherein
the biosample processing apparatus further includes: a tubular discharge path connected to the other end of the flow path; and a second switch part provided at a connection part of the separation part between the flow path and the discharge path, and
the biosample processing method further comprises:
selectively switching the second switch part to a direction of flow from the separation part to the second collection part or a direction of flow from the separation part to the discharge path; and
discharging the first sample after the second sample is separated from the discharge path when the second switch part is switched to the direction of flow from the separation part to the discharge path.

14. The biosample processing method according to claim 12, wherein
the biosample processing apparatus further includes a third collection part connected to the first switch part, and
the biosample processing method further comprises:
selectively switching the first switch part to a direction of flow from the second collection part to the culture part or a direction of flow from the separation part to the third collection part; and
collecting part of the second sample as a test sample to the third collection part when the first switch part is switched to the direction of flow from the separation part to the third collection part.

15. The biosample processing method according to claim 14, further comprising:
acquiring an analysis result of components of the test sample; and
determining a supply amount of the second sample to the culture part based on the analysis result.
